(19) United States
(12) Patent Application Publication
(10) Pub. No.: US 2017/0296096 A1
(43) Pub. Date: Oct. 19, 2017

(54) WEARABLE DEVICE AND METHOD FOR DETECTING REFLECTIVE ZONES USING SENSORS

(71) Applicants: Carolyn Taylor, Washington, DC (US); Angie Taylor, Washington, DC (US)

(72) Inventors: Carolyn Taylor, Washington, DC (US); Angie Taylor, Washington, DC (US)

(73) Assignee: Hepta 7291 LLC, Washington, DC (US)

(21) Appl. No.: 15/097,395

(22) Filed: Apr. 13, 2016

(51) Int. Cl. A61B 5/103; H04W 4/00; A61B 5/00; H04B 5/00; H04B 1/3827; A61B 90/00

(52) U.S. Cl. CPC A61B 5/1038; H04W 4/008; H04W 4/005; A61B 5/6807; A61B 5/6893; A61B 5/7282; A61B 5/7285; A61B 5/7465; H04B 5/0037; H04B 1/385; A61B 2560/0475; A61B 2562/0247; A61B 2562/221; A61B 2560/0209; A61B 2560/0214; A61B 2090/064

(57) ABSTRACT

On a wearable and Internet of Things (IOT) device that is proximate to a user equipment, a method to detect reflective zones of the wearable device may include, in response to a preset criterion, obtaining a characteristic of the left foot pressure points, obtaining a characteristic of the right foot pressure points, obtaining a pressure sensor on the wearable device, obtaining a position sensor on the wearable device, obtaining a force sensor on the wearable device, obtaining kinetic energy movement from the wearable device, and obtaining the radio characteristic of the wearable device. The method may also include transmitting the left foot pressure points characteristic, the right foot pressure points characteristic, the pressure sensor on the wearable device, the position sensor on the wearable device, the force sensor on the wearable device, the kinetic energy movement from the wearable device, and the radio characteristic to a user equipment.

### TECHNICAL FIELD OF THE INVENTION

The present disclosure relates to a wearable and Internet of Things (IoT) device. More particularly, the present disclosure relates to a wearable device wearable on a user's body, for generating a stimulus according to an operation state of the device, and a method for detecting reflective zones of the wearable device.

### BACKGROUND OF THE INVENTION

Currently, devices wearable by the user's body whilst performing as an assortment of smart functions have been developed. Wireless communication is continuously evolving. There are many types of advanced technology equipment being introduced that can provide services that were not possible previously. This advanced technology equipment might include, for example, an Enhanced Node B (eNB) or other systems and devices that are more highly evolved than the equivalent equipment in a traditional wireless telecommunications system. Such advanced or next generation equipment may be referred to herein as LTE, LTE-Advanced, whose networks are also known as Evolved Universal Terrestrial Access Network (E-UTRAN), is a technology that can reach high data rates both in the downlink as well as in the uplink. LTE allows for a system bandwidth of 20 MHz, or up to 100 Hz with certain features. It uses the Global Navigation Satellite System (GLASS) for gathering positioning information, which could include radio access technology (RAT) independent positioning methods such as: barometrics, sensor method, WLAN method, Bluetooth method, or Terrestrial Beacon System Method. Representative examples of the wearable devices may include a smart band, a smart glass, a smart eye, a smart watch, and the like. Examples of wearable devices can provide a variety of functions for making a call like a wireless device, sending text messages or email, web surfing, and the like. Devices with wireless communications capabilities, such as mobile telephones, handheld devices, Machine-2-Machine devices (M2M), Device-to-Device (D2D) communication devices, and similar devices, will be referred to herein as User Equipment (UE) or wireless devices.

A heterogeneous network (HetNet) is a network that includes infrastructure points with various wireless access technologies, each of them having different capabilities, constraints, and operating functionalities. A typical HetNet includes a mix of macro-cells and low-power nodes such as picocells, femtocells, and relays, to name a few. Small cells are low powered radio access nodes that can operate in licensed and unlicensed spectrum. A typical small cell include femtocells, picocells, and microcells which broadly increase in size from femtocells (the smallest) to microcells (the largest). Any or all of these small cells can be based on femtocell technology.

Leveraging network topology, increasing the proximity between the access network and the end-users, has the potential to provide the next significant performance leap in wireless networks.

Different UEs might use different types of radio access technology (RAT) to access a wireless communications network. Some UEs, referred to as multi-mode UEs, are capable of communicating using more than one RAT. For example, multi-mode UEs may include UEs that can obtain service from at least one mode of UMTS, and one or more different technologies such as GSM (Global System for Mobile Communications) or other radio systems. As defined herein, multi-mode UEs may be of any various type of multi-mode UE as defined or provided in 3GPP, Technical Specification Group (TSG) Terminals, Multi-Mode UE Issues, Categories, Principles and Procedures (3G TR 21.910). Some examples of RATs or network technologies that might use different types of RATs include Evolved Universal Terrestrial Radio Access (E-UTRA), UT RAN (UTMS Terrestrial Radio Access Network), GSM, GSM Enhanced Data rates for Global Evolution (EDGE) Radio Access Network (GERAN), Wireless Fidelity (WiFi), Bluetooth (BT), General Packet Radio Service (GPRS), High-Speed Downlink Packet Access (HSDPA), HSPA, and LTE. Other RATs or other network technologies based on these RATs may be familiar to one of skill in the art. It also uses GNSS (including GPS, Galileo, GLONASS, Beidou) with RATs for the User Equipment (UE).

Reflexology of the foot may be a medial indicator, easy detection of diseases and leading indicator of their prompt correction. It is perhaps a natural therapy through which one can easily ascertain the normal or abnormal functioning of different internal organ of the body instantly by pressing certain reflex points in hands and feet. These reflex points on the feet will trigger the change inside your body and can possibly correct the problem. Examples of bodily events, the outside of the foot, the bottom of the feet has reflex points about buttock, knee, elbow, arm and shoulders. Another example is that in a similar way the toes carries the problems related to the head and in the middle of the feet you have reflex points which help with problems of the back and of the eye. These important points if given the right amount of pressure can trigger the real change inside the body.

The feet are full of reflexology points. In case of danger, the brain communicates with the internal organs through the autonomic nervous system and the muscles, such as those of the foot, through the central nervous system. The reflexive response to a pressure stimulus also requires a response by the autonomic nervous system and the central nervous system. The feet are not mirror images of each other when it comes to reflexology, although they share many characteristics.

One or more pressure stimulus indicators may be associated with abnormal functioning of different internal organ of the body and be measured using a variety of sensors and systems, such as metrics from left foot pressure points, right foot pressure points, left hand pressure points, right hand pressure points, just to name a few. For example, applying pressure to a spot on the arch of the foot using sensors may benefit bladder function.

Moreover, environmental, dietary, social conditions, and other externalities may impact bodily functions that cause abnormal functioning of different internal organ of the body, such as stress from work, school, relationships, family members, finances, commuting, etc. Reflexology of the foot may be used as a treatment that may be helpful as a tool to help identify and diagnose medical conditions or diseases or as an indicator of early onset of disease. Ideally, it is primarily used for ongoing (chronic) pain, especially cancer-related pain. It is also used for asthma, lung disease (chronic obstructive pulmonary disease, COPD), chest pain (angina), back pain, constipation, children's inability to control bowel movements (encopresis), fibromyalgia, headache, migraine, multiple sclerosis (MS), arthritis, overactive bladder, and stress. Some women use reflexology to treat symptoms of menopause and premenstrual syndrome (PMS), just to name a few.

### SUMMARY OF THE INVENTION

An aspect of the present invention provides for a user wearable device that detects a plurality of bodily metrics, reflective zones and external data to measure pressure points that indicate normal or abnormal functioning of different internal organs of the body and to provide this information to the user, and to optionally provide coaching and/or remediation to the user. The wearable device will also be connected to vehicles and have the ability to use the kinetic energy from the vehicle movement to charge the wearable device.

Another aspect of the present invention is to provide a wearable device that is proximate to a user equipment, a method to detect reflective zones of the wearable device may includes in response to a preset criterion, obtaining a characteristic of the left foot pressure points, obtaining a characteristic of the right foot pressure points, obtaining a pressure sensor on the wearable device, obtaining a position sensor on the wearable device, obtaining a three sensor on the wearable device, obtaining kinetic energy of your movement from the wearable device, and obtaining the radio characteristic of the wearable device. The method may also include transmitting the left foot pressure points characteristic, the right foot pressure points characteristic, the pressure sensor on the wearable device, the position sensor on the wearable device, the force sensor on the wearable device, the kinetic energy of your movement from the wearable device, and the radio characteristic to a user equipment.

In another embodiment of the present invention, a method includes detecting a trigger measuring condition and carrying out the obtaining steps and the transmitting step in response to detecting the trigger measuring condition.

In another embodiment of the present invention, a trigger measuring condition may be the wearable device moving away from a serving cell and toward the user equipment.

In another embodiment of the present invention, the criterion may be a single event or a periodical event.

In another embodiment of the present invention, the method further includes repeating the obtaining of the left foot pressure points characteristics, obtaining of the right foot pressure points characteristics, obtaining of the pressure sensor on the wearable device, obtaining of the position sensor on the wearable device, obtaining of the force sensor on the wearable device, harnessing the kinetic energy of the user's movement from the wearable device, and obtaining the radio characteristic of the user's equipment.

In another embodiment of the present invention, the sensors of the wearable device are the characteristic of the pressure sensor on the wearable device, the position sensor on the wearable device, the force sensor on the wearable device, and the kinetic energy movement from the wearable device.

In another embodiment of the present invention, the reflective zones of the wearable device are of the left foot and the characteristic is the left foot pressure points face on the wearable device.

In another embodiment of the present invention, the reflective zones of the wearable device are of the right foot and the characteristic is the right foot pressure points face on the wearable device.

In another embodiment of the present invention, a method for detecting the reflective zones of the wearable device is carried out on user equipment. The method includes requesting a report from the wearable device. In response to the request from the wearable device is received a report comprising a characteristic of wearable device that is proximate to the user equipment, a characteristic of the left foot pressure points of the wearable device, a characteristic of the right foot pressure points of the wearable device, the pressure sensor on the wearable device, the position sensor on the wearable device, the force sensor on the wearable device, kinetic energy movement from the wearable device, and a radio characteristics of the wearable device. The impact of the left foot pressure points characteristic, the right foot pressure points characteristic, the pressure sensor on the wearable device, the position sensor on the wearable device, the force sensor on the wearable device, the kinetic energy movement from the wearable device, and the radio characteristics of the wearable device are determined. The reflective zones in light of the left foot pressure points characteristic, the right foot pressure points characteristic, the pressure sensor on the wearable device, the position sensor on the wearable device, the force sensor on the wearable device, the kinetic energy movement from the wearable device, and the radio characteristics of the wearable device are calculated. An updated left foot pressure points characteristic, an updated right foot pressure points characteristic, an updated pressure sensor on the wearable device, an updated position sensor on the wearable device, an updated force sensor on the wearable device, an updated kinetic energy movement from the wearable device, and an updated radio characteristic of the wearable device are detected or obtained.

In another embodiment of the present invention, the method for determining the pressure points of equipment wearable device also includes repeating the determining, calculating and the detecting or obtaining steps until pressure points are obtained.

In another embodiment of the present invention, the sensors of the wearable device that sense the characteristic are the pressure sensor on the wearable device, the position sensor on the wearable device, the force sensor on the wearable device, and the kinetic energy movement from the wearable device.

In another embodiment of the present invention, the reflective zones of the wearable device are of the right foot and the characteristic is the right foot pressure points on the wearable device.

In another embodiment of the present invention, the reflective zones of the wearable device are of the right foot and the characteristic is the right foot pressure points on the wearable device.

In another embodiment of the present invention, a wearable device includes sensors, antennas, charger ports, low powered battery, wireless communications, a memory, and a processor. The processor retrieves instructions from the memory and executes the instructions to (in response to a preset criterion) obtain a characteristic of the left foot pressure points, obtain a characteristic of the right foot pressure points, obtain a pressure sensor on the wearable device, obtain a position sensor on the wearable device, obtain a force sensor on the wearable device, harness the kinetic energy movement from the wearable device, and obtain the radio characteristic of the wearable device. Further, the method includes transmitting the left foot pressure points characteristics, the right foot pressure points characteristics, the pressure sensor on the wearable device, the position sensor on the wearable device, the force sensor on the wearable device, the kinetic energy movement from the wearable device, and the radio characteristic to user equipment.

In some embodiments the processor also detects a trigger measuring condition, carries out the obtaining steps and the transmitting step in response to detecting the trigger measuring condition.

In some embodiments, the trigger measuring condition is the wearable device moving away from a serving cell and toward the user equipment.

In some embodiments, the processor repeats the obtaining of the left foot pressure points characteristic, the obtaining of the right foot pressure points characteristic, the obtaining of the pressure sensor on the wearable device, the obtaining of the position sensor on the wearable device, the obtaining of the force sensor on the wearable device, and the obtaining of the radio characteristic of the user equipment.

In some embodiments, a low powered battery captures the kinetic energy of your movement powering the wearable device.

In some embodiments, the battery charger status is captured on the wearable device.

In some embodiments, the wearable device will also be connected to vehicles and have the ability to use the harnessed kinetic energy from the vehicle movement and bumps to charge the wearable device.

### BRIEF DESCRIPTION OF THE DRAWINGS

While the appended claims set forth the features of the present techniques with particularity, these techniques, together with their objects and advantages, may he best understood from the following detailed description taken in conjunction with the accompanying drawings of which:

FIG. 1 is a block diagram of a wearable device according to an embodiment of the present invention.

FIG. 2 is a block diagram of a system according to an embodiment of the present invention.

FIG. 3 is a diagram of a left foot insole/shoe insert according to an embodiment of the present invention.

FIG. 4 is a diagram of a right foot insole/shoe insert according to an embodiment of the present invention.

FIG. 5 is a flowchart of a method for measuring signal strength according to an embodiment of the present invention.

FIG. 6 is a flowchart of a method for obtaining the left foot pressure points, right foot pressure points, pressure sensor on the wearable device, position sensor on the wearable device, force sensor 011 the wearable device, and radio characteristics of information according to an embodiment of the present invention.

FIG. 7 is a flowchart of a method for obtaining the left foot pressure points, right foot pressure points, pressure sensor on the wearable device, position sensor on the wearable device, force sensor on the wearable device, and radio characteristics of information according to another embodiment of the present invention.

FIG. 8 is a diagram that illustrates an example of a trigger measuring condition according to an embodiment of the present invention.

FIG. 9 is a diagram of a data transmission according to an embodiment of the present invention.

FIG. 10 is a flowchart of a method for obtaining of the left foot pressure points characteristic, the right foot pressure points characteristic, the pressure sensor on the wearable device, the position sensor on the wearable device, the force sensor on the wearable device, and the radio characteristic of the user equipment according to an embodiment of the present invention.

FIG. 11 depicts a profile view of pressures, positions and forces acting on a user having a wearable device to detect reflective points according to an embodiment of the present invention.

FIG. 12 depicts a block diagram of sensor systems, data communication systems, data processing systems, user equipment, and data systems that may be coupled with and/or in communication with a wearable device to detect reflective points according to an embodiment of the present invention.

FIG. 13 depicts examples of sensor inputs and/or data that may be sourced internally or externally in a wearable device to passively detect reflective points of a user according to an embodiment of the present invention.

FIG. 14 is a block diagram illustrating a structure of a wearable device according to an embodiment of the present invention.

FIG. 15 is a diagram for explanation of a structure of software stored in a storage according to an embodiment of the present invention,

FIG. 16 is a diagram of the foot insole/shoe insert reflexology face layer 3 according to an embodiment of the present invention.

FIG. 17 is a diagram of the foot insole/shoe insert sensors layer 2 according to an embodiment of the present invention.

FIG. 18 is a diagram of the foot insole/shoe insert technology layer 1 that includes charger ports, low powered battery, wireless communications, flexible sensor cable, processors, memory, and antennas according to an embodiment of the present invention.

FIG. 19 is a diagram of the wearable device connected to vehicles that have the ability to use the harness kinetic energy from the vehicle movement and bumps to charge the wearable device according to an embodiment of the present invention.

### DETAILED DESCRIPTION OF THE PREFERRED EMBODIMENTS

According to various embodiments, a method to detect reflective zones of the wearable device using the left foot, pressure points characteristics, the right foot pressure points characteristics, pressure sensor on the wearable device, position sensor on the wearable device, force sensor on the wearable device, and radio characteristics of the wearable device is proposed. A system is provided for measuring the reflective zones of the wearable device using the left foot pressure points characteristics, the right foot pressure points characteristics, pressure sensor on the wearable device, position sensor on the wearable device, force sensor on the wearable device, the kinetic energy movement from the wearable device, radio characteristics of the wearable device is proposed. The system may include left foot insole/shoe insert, right foot insole/shoe insert, pressure sensor, position sensor, force sensor, and a user equipment (UE). The UE may be a multi-mode UE that is capable of communicating via multiple RATs. The multimode UE can include a processor configured to promote measurements of a signal strength in a communication system.

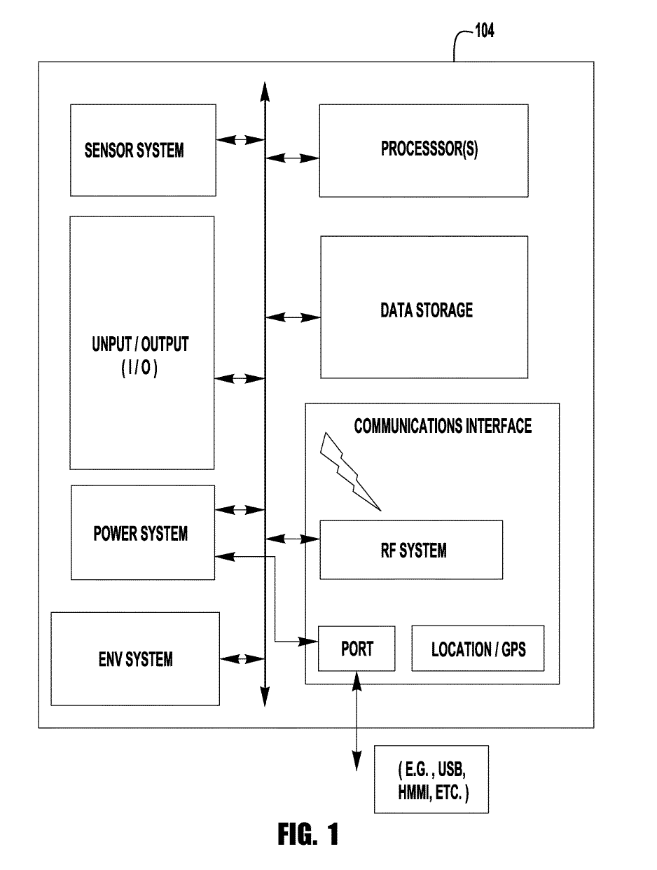

FIG. 1 depicts a block diagram of a wearable device.

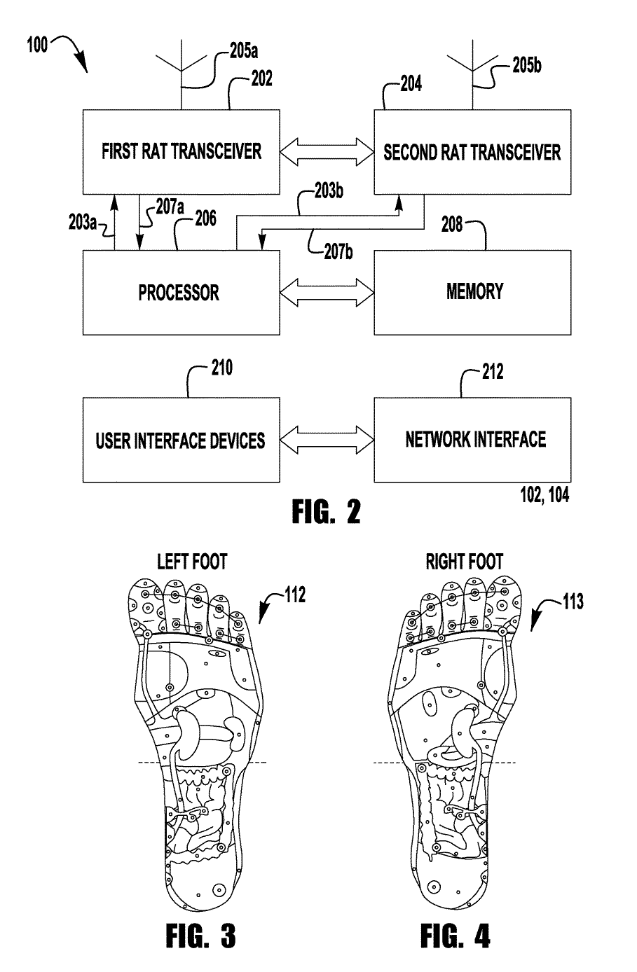

An exemplary embodiment of this system is shown in FIG. 2. In this embodiment, a system 100 that may be used to carry out various embodiments is generally labeled 100.

During operation of the user equipment 102 or the wearable device 104, one or more of the transceivers 202 and 204 receives data through lines 203a and 203b from the processor 206 and transmits radio-frequency signals through the antennas 205a and 205b representing the data. Similarly, one or more of the transceivers 202 and 204 receives radio-frequency signals, converts the signals into the appropriately formatted data, and provides the data to the processor 206 through lines 207a and 207b. The processor 206 retrieves instructions from the memory 208 and, based on those instructions, provides outgoing data to one or more of the transceivers 202 and 204 or receives incoming data from the one or more of the transceivers 202 and 204. Similarly, based on the instructions, the processor 206 carries out one or more of the various methods disclosed herein, such as making the various measurements discussed herein, transmitting the various reports discussed herein in order to promote the measurement of a signal strength in a communication system.

The processor 206 may be any programmable device such as a computer, a microprocessor, a microcontroller, a set of peripheral integrated circuit elements, an integrated circuit (e.g., an application-specific integrated circuit), hardware/electronic logic circuits (e.g., a discrete element circuit), a programmable logic device (e.g., a programmable logic array), or a field programmable gate-array.

Possible implementation of the memory 208 include volatile memory, non-volatile memory, electrical, magnetic optical memory, random access memory (RAM), cache, and hard disc.

Turning to FIG. 3, an embodiment of the left foot insole/shoe insert 112 is shown.

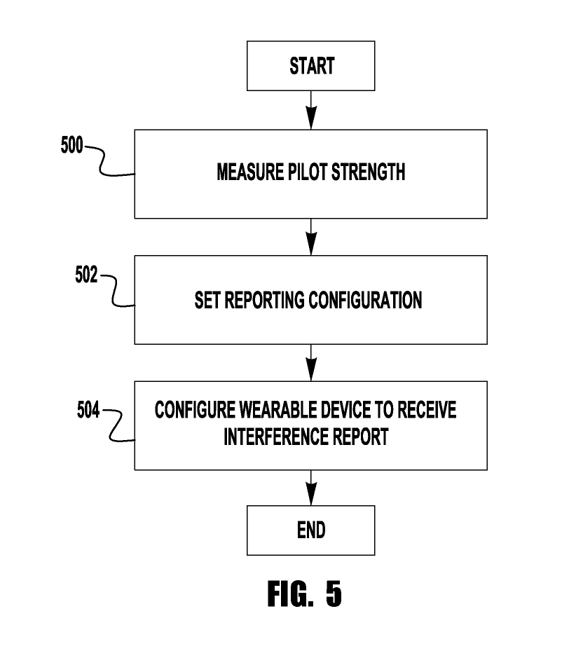

Turning to FIG. 4, another embodiment of the right foot insole/shoe insert 113 is shown.

Turning to FIG. 5, a method that is carried out-e.g., by the wearable device 104, to measure signal strength in an embodiment will now be described. At block 500, a pilot strength is measured. For example, a UE may measure the strength of a pilot signal from an advanced technology network (e.g., an LTE network). At block 502, a reporting configuration for the wearable device 104 is set. For example, the wearable device 104 is configured to report one or more characteristics of the left foot insole/shoe insert 112, right foot insole/shoe insert 113, a characteristic (or characteristics) of one or more of the sensors of the wearable device 104, and one or more radio characteristics being experienced by the wearable device 104. A preset criterion that triggers the wearable device 104 to send the report may be a periodical event or a single event. At block 504, the wearable device 104 is configured to receive an interference report. For example, the wearable device (such as the wearable device 104) is configured to receive a report to inform a serving cell (such as the serving cell 114) to inform the serving cell of the interference capability of the UE based on measurements received by the UE from the serving cell and a neighboring cell (such as the neighboring cell 116).

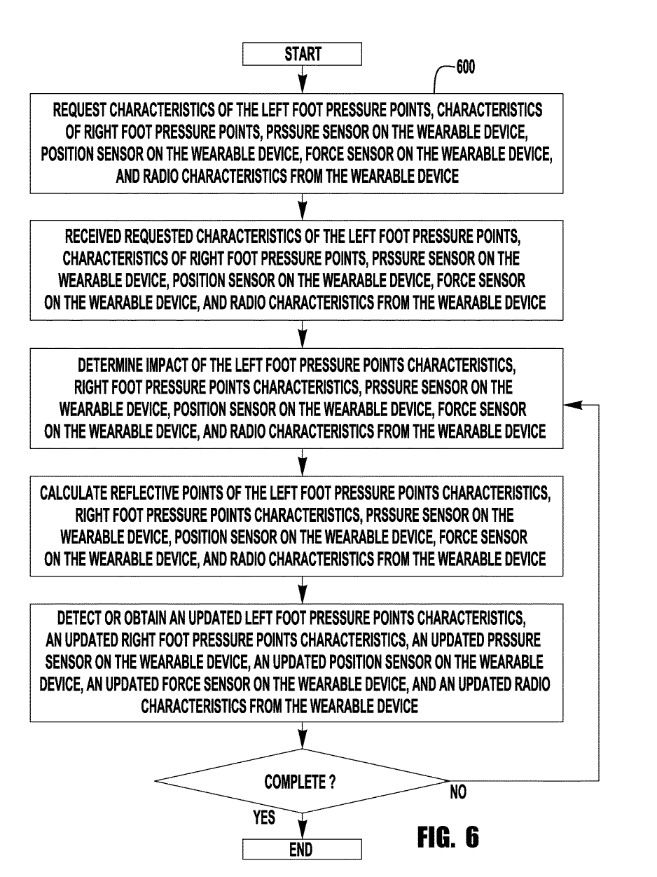

Turning to FIG. 6, a method that is carried out by the user equipment 102 to obtain left foot pressure points characteristics, the right foot pressure points characteristics, pressure sensor on the wearable device, position sensor on the wearable device, force sensor on the wearable device, radio characteristics from the wearable device 104 according to an embodiment will now be described. At block 600, the user equipment 102 requests left foot pressure points characteristics, the right foot pressure points characteristics, pressure sensor on the wearable device, position sensor on the wearable device, force sensor on the wearable device, radio characteristics from the wearable device 104.

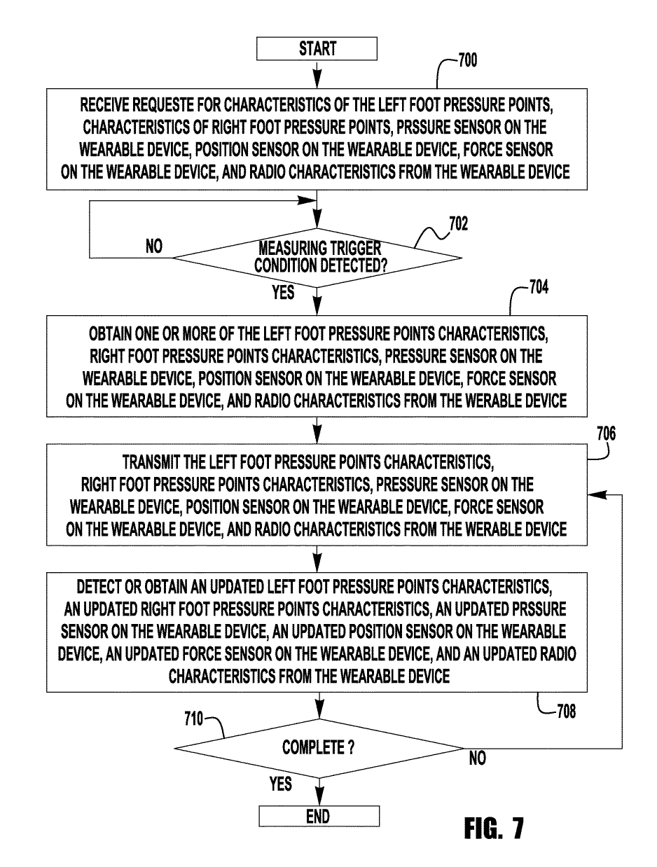

Turning to FIG. 7, a method that is carried out by the wearable device 104 to obtain obtaining the left foot pressure points, right foot pressure points, pressure sensor on the wearable device, position sensor on the wearable device, force sensor on the wearable device, and radio characteristics according to an embodiment will now be described. At block 700, the UE 104 receives a request—e.g., from the user equipment 102—for left foot pressure points, right foot pressure points, pressure sensor on the wearable device, position sensor on the wearable device, force sensor on the wearable device, and radio characteristics. Otherwise, the wearable device 104 waits. At block 702, if the wearable device 104 detects a trigger measuring condition, it moves to block 704. At block 704, the wearable device 104 obtains one or more characteristics of the left foot insole/shoe insert 112, right foot insole/shoe insert 113, measure its own radio characteristics, and measures characteristics of one or more of the user equipment 102. At block 706, the wearable device 104 transmits the left foot pressure points, right foot pressure points, pressure sensor on the wearable device, position sensor on the wearable device, force sensor on the wearable device, and radio characteristics to the user equipment 102. In some embodiments, the configuration of the wearable device 104 is changed after block 706. For example, the configuration of the wearable device 104 is may be changed between a data communication mode and a speech communication mode.

Turning to FIG. 8, an example of a trigger measuring condition (i.e., one that may occur when a measurement criterion has been fulfilled) will now be described. In this example, the measurement criterion is that a UE 800 (which could be the wearable device 104 of FIG. 1) is receiving data, in the form of a data transmission 801 from an eNB of a macro technology network 802 (which could be the serving cell 114) and is moving away from the macro technology network 802 but closer to a micro technology network 804 (which could be the user equipment I 02). Thus, when the UE 800 determines that it is moving away from the macro technology network 802 but closer to the micro technology network 804, the HE 800 responds by (i.e., is triggered to) carry out the steps set forth in blocks 704 through 710 of FIG. 7.

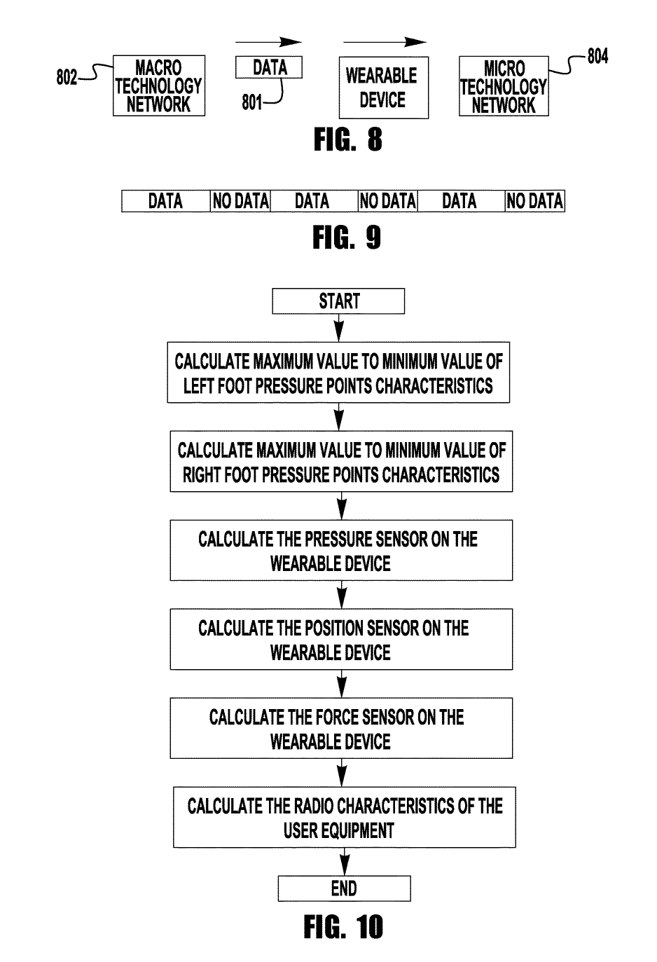

FIG. 9 illustrates a detailed view of a data transmission that a UE may send according to an embodiment. The data transmission includes a series data strings separated by transmission period in which no data is transmitted. The data strings might represent some type of a user directed data transmission. During the period in which no data is transmitted, the UE can measure the strengths of the signals that it receives.

Turning to FIG. 10, a method for obtaining of the left foot pressure points characteristic, the right foot pressure points characteristic, the pressure sensor on the wearable device, the position sensor on the wearable device, the force sensor on the wearable device, and the radio characteristic of the user equipment.

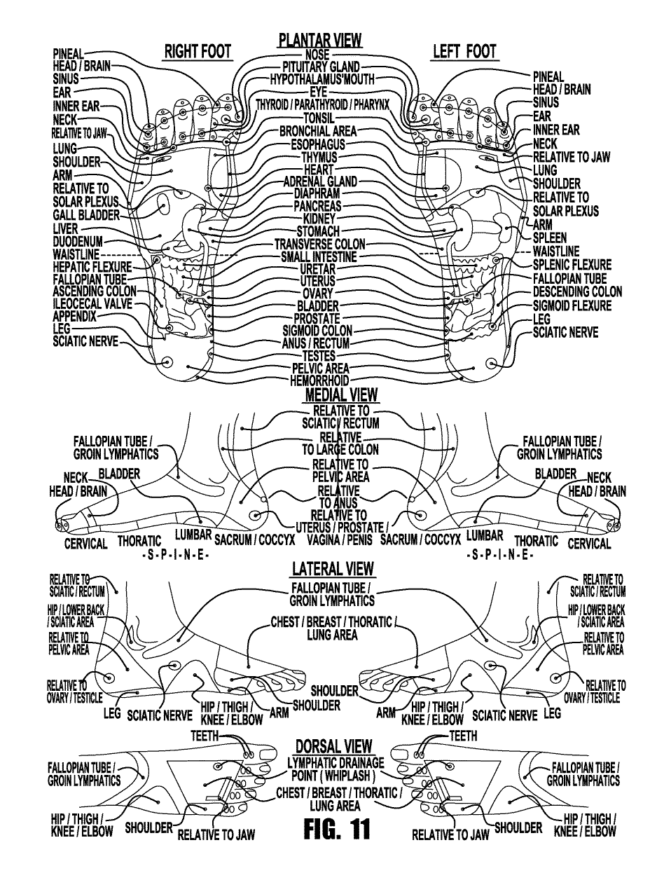

FIG. 11 depicts an embodiment of a method for a profile view of pressures, positions and forces acting on a user having a wearable device to detect reflective points.

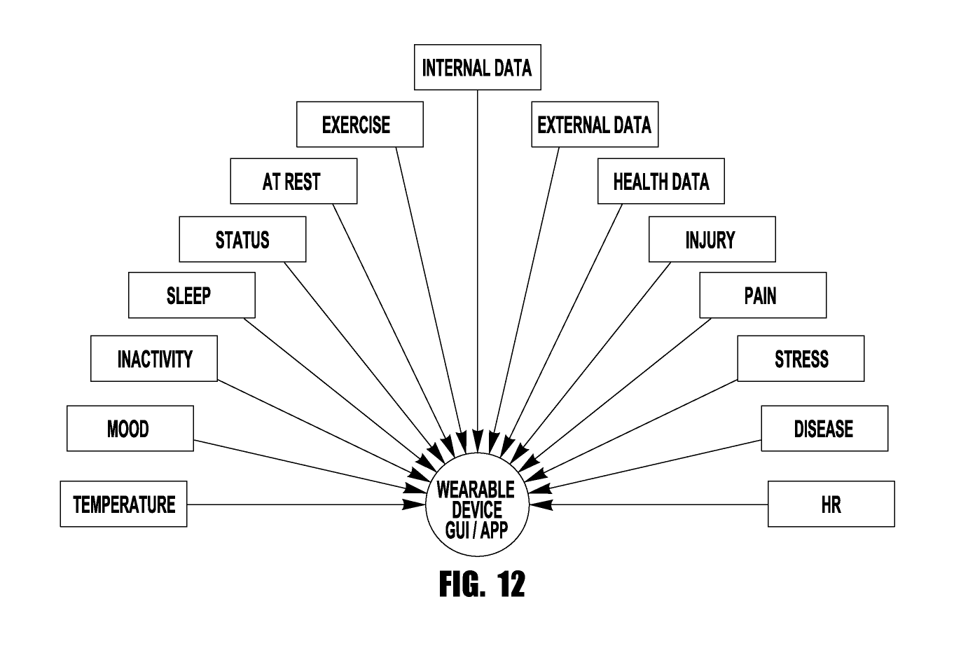

FIG. 12 depicts an embodiment of a method for a block diagram of an example of a cycle of monitoring a user having a wearable device to detect reflective points and data inputs that may be used in a calculus for determining normal or abnormal functioning of different internal organ of the body instantly by pressing certain reflex points in hands and feet, which are indicated in the user.

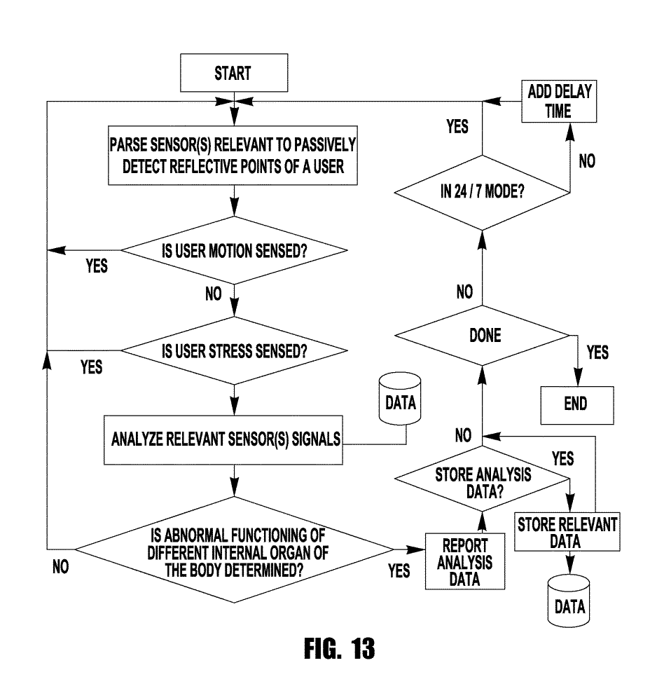

FIG. 13 depicts an embodiment of an example of sensor inputs and/or data that may be sourced internally or externally in a wearable device to passively detect reflective points of a user.

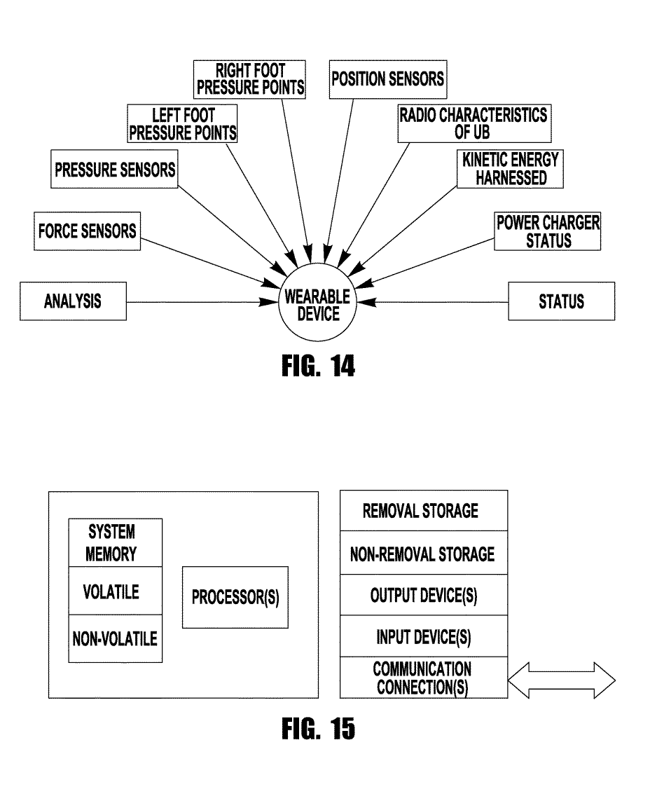

FIG. 14 depicts an embodiment of a method for a block diagram illustrating a structure of a wearable device.

FIG. 15 depicts an embodiment of a diagram for explanation of a structure of software stored in a storage.

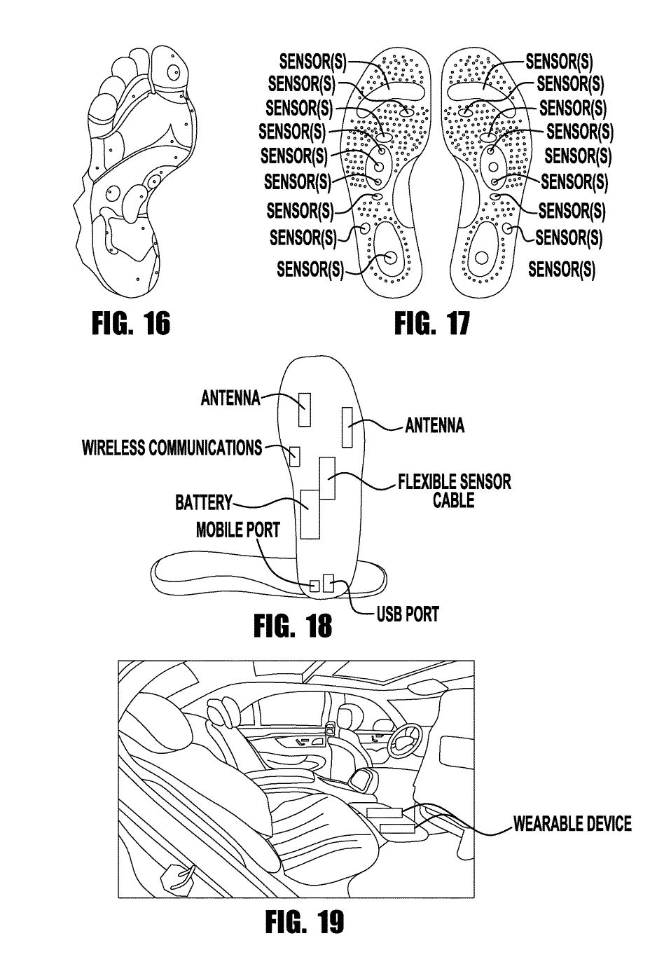

FIG. 16 depicts an embodiment of a diagram for the foot insole/shoe insert reflexology face layer 3.

FIG. 17 depicts an embodiment of a diagram for the foot insole/shoe insert sensors layer 2.

FIG. 18 depicts an embodiment of a diagram for the foot insole/shoe insert technology layer 1 that includes charger ports, low powered battery, wireless communications, flexible sensor cable, processors, memory, and antennas.

FIG. 19 depicts an embodiment of a diagram for the wearable device connected to vehicles that have the ability to use the harness kinetic energy from the vehicle movement and bumps to charge the wearable device.

In various embodiments, the method includes creating a criterion that triggers a multi•mode UE to send a measurement report. This can either be periodical or a single event description.

In various embodiments, the above method includes a wearable and Internet of Things (IoT) device for obtaining the left foot pressure points, right foot pressure points, pressure sensor on the wearable device, position sensor on the wearable device, force sensor on the wearable device, the kinetic energy movement from the wearable device, and radio characteristics for detecting reflective zones of the wearable device.

In various embodiments, the wearable device to be measured is arranged at the position as a complete coupling position based on the wearable device to detect reflective points, left foot pressure points characteristics, right foot pressure points characteristics, pressure sensor, position sensor, force sensor, radio characteristics; the left foot pressure points characteristic, the right foot pressure points characteristic, the pressure sensor on the wearable device, the pressure sensor on the wearable device, the force sensor on the wearable device, the kinetic energy movement from the wearable device, and the radio characteristics of the wearable device to be measured is calculated.

In various embodiments, the wireless device is held in the data communication mode.

While one or more embodiments of the have been described with reference to the figures; it will be understood by those of ordinary skill in the art that various changes in form and details may be made therein without departing from their spirit and scope of as defined by the following claims. For example, the steps of the flowcharts of FIGS. 5, 6, 7, and 10 can be reordered in way that will be apparent to those of skill in the art.

1. A method to detect reflective zones of a left foot and a right foot with a wearable device, comprising:
providing the wearable device with a left foot and a right foot wearable device;
in response to a preset criterion, detecting a first characteristic signal of left foot pressure points with the left foot wearable device;
in response to a preset criterion, detecting a second characteristic signal of right foot pressure points with the right foot wearable device;
detecting first and second pressure signals with the left and right foot wearable devices, respectively;
detecting first and second kinetic energy movement signals with the left and right foot wearable devices, respectively;
charging the left and right wearable devices with kinetic energy generated from movement of a vehicle to charge the wearable device,
obtaining a radio characteristic of the wearable device; and
transmitting the first characteristic signal of the left foot pressure points, the second characteristic signal of the right foot pressure points characteristic, the first and second pressure signals of the left and right foot wearable devices, the first and second kinetic energy movement signals from the left and right foot wearable devices, and the radio characteristic of the wearable device to user equipment.

2. The method claim 1, further comprising:
detecting a trigger measuring condition; and
transmitting the first characteristic signal, the second characteristic signal, the first and second pressure signals, the first and second kinetic energy movement signals, and the radio characteristic to the user equipment in response to detecting the trigger measuring condition.

3. The method claim 2, wherein detecting the trigger measuring condition occurs when the wearable device is moving away from a serving cell and toward the user equipment.

4. The method of claim 2, including selecting a single event as the response to the preset criterion.

5. The method of claim 2, including selecting a periodic event as the response to the preset criterion.

6. The method of claim 1, further comprising repeating steps of:
detecting the first and second characteristic signals of the left and right foot pressure points;
detecting the first and second pressure signals with the left and right foot wearable devices;
detecting first and second kinetic energy movement signals; and
detecting the radio characteristic for a plurality of different configurations of the wearable device relative to the reflective zones.

7. The method of claim 6, including configuring the wearable device by positioning the wearable device in a plurality of different positions of the wearable device relative to the reflective zones.

8. The method of claim 6, including:
selecting the detected reflective zones from the left foot pressure points; and
configuring the plurality of different configurations of the wearable device by moving the wearable device to a plurality of different positions relative to the left foot pressure points on the wearable device.

9. The method of claim 6, wherein:
selecting the detected reflective zones are selected from of the right foot pressure points; and
configuring the plurality of different configurations of the wearable device by moving the wearable device to a plurality of different positions relative to the t foot pressure points on the wearable device.

10. The method of claim 6, wherein the configuration of the wearable device comprises a data communication mode and a speech communication mode.

11. The method of claim 1, including:
detecting the reflective zones as the left foot pressure points; and
selecting the characteristic signals from the pressure points on the wearable device.

12. The method of claim 1, including:
detecting the reflective zones as the right foot pressure points; and
selecting the characteristic signals from the pressure points on the wearable device.

13. The method of claim 1, including selecting the reflective zones as the pressure sensor on the wearable device.

14. The method of claim 1, including selecting the reflective zones as the position sensor on the wearable device.

15. The method of claim 1, including selecting the reflective zones as a force sensor on the wearable device.

16. The method of claim 1, including selecting the reflective zones used to harness kinetic energy movement to the wearable device.

17. On a user equipment, a method for determining the reflective points of a wearable device, the method comprising:
requesting a report from the wearable device that is proximate to the user equipment;
in response to the request, receiving from the wearable device, the report including characteristics of left foot pressure points, characteristics of right foot pressure points, characteristics corresponding to a pressure sensor on the wearable device, characteristics corresponding to a position sensor on the wearable device, characteristics corresponding to a force sensor on the wearable device, characteristics corresponding to kinetic energy movement from the wearable device, and radio characteristics of the wearable device;
determining the impact of the left foot pressure points characteristics, the right foot pressure points characteristics, the characteristics of the pressure sensor on the wearable device, the characteristics of the position sensor on the variable device, the characteristics of the force sensor on the wearable device, the kinetic energy movement from the wearable device, and the radio characteristics from the wearable device;
calculating the reflective points of the left foot pressure points characteristics, the right foot pressure points characteristics, the characteristics of the pressure sensor on the wearable device, position sensor on the wearable device, the characteristics of the force sensor on the wearable device, the kinetic energy movement from the wearable device, and the radio characteristics from the wearable device; and
detecting an updated left foot pressure point characteristics, an updated right foot pressure point characteristics, an updated characteristics of the pressure sensor on the wearable device, an updated position sensor on the wearable device, an updated characteristics of the force sensor on the wearable device, an updated kinetic energy movement from the wearable device, and an updated radio characteristics from the wearable device.

18. The method of claim 17, further comprising repeating the determining, calculating and the detecting steps until complete.

19. The method of claim 17, including selecting the reflective zones as the left foot pressure points and the characteristics as the pressure points on the wearable device.

20. The method of claim 17, including selecting the reflective zones is the right foot pressure points and the characteristics as the pressure points on the wearable device.

21. The method of claim 17, including selecting the reflective zones as the pressure sensor on the wearable device.

22. The method of claim 17, including selecting the reflective zones as the position sensor on the wearable device.

23. The method of claim 17, including selecting the reflective zone as the force sensor on the wearable device.

24. The method of claim 17, including harnessing kinetic energy movement to the wearable device from the force sensor on the wearable device.

25. A wearable device comprising:
antennas;
a memory;
charge ports;
flexible sensor cable;
low powered battery;
wireless communications; and
a processor that receives instructions from the memory and executes the instructions to carry out steps comprising, in response to a preset criterion, means for obtaining a first characteristic signal of the left foot pressure points, means for obtaining a second characteristic signal of the right foot pressure points, obtaining a pressure signal from a pressure sensor on the wearable device, means for obtaining a position signal from a position sensor on the wearable device, means for obtaining force signal from a force sensor on the wearable device, means for obtaining a kinetic energy movement signal from the wearable device, means for harnessing the kinetic energy from movement of a vehicle to charge the wearable device, means for obtaining a radio characteristic signal of the wearable device; and
means for transmitting the left foot pressure points characteristic, the right foot pressure points characteristic, the pressure signal from the pressure sensor on the wearable device, the position signal from the position sensor on the wearable device, the force signal from the force sensor on the wearable device, the kinetic energy movement signal from the wearable device, and the radio characteristic signal to a user equipment.

26. The wearable device of claim 25, further comprising:
means for detecting a trigger measuring condition; and
means for carrying out the obtaining steps and the transmitting step in response to detecting the trigger measuring condition.

27. The wearable device of claim 25, wherein the trigger measuring condition is the wearable device moving away from a serving cell and toward the user equipment.

28. The wearable device of claim 25 wherein battery charger status is captured on the wearable device.

29. The wearable device of claim 25 wherein the harness kinetic energy from the movement of the vehicle charges the wearable device.
